(19) United States
(12) Patent Application Publication
(10) Pub. No.: US 2016/0144032 A1
(43) Pub. Date: May 26, 2016

(54) COMPOSITION FOR NEW BONE FORMATION, AND NEW BONE FORMATION SYSTEM

(71) Applicant: NATIONAL UNIVERSITY CORPORATION NAGOYA UNIVERSITY, Aichi (JP)

(72) Inventors: Yoshihiro NISHIDA, Aichi (JP); Kunihiro IKUTA, Aichi (JP); Hiroyuki HONDA, Aichi (JP); Ryuji KATO, Aichi (JP); Takeshi KOBAYASHI, Aichi (JP)

(21) Appl. No.: 14/900,040

(22) PCT Filed: Jun. 5, 2014
(86) PCT No.: PCT/JP2014/064920

§ 371 Date: Dec. 18, 2015

(30) Foreign Application Priority Data
Jun. 18, 2013 (JP) 2013-128021

(51) Int. Cl. A61K 41/00; A61L 27/52; A61F 2/28; A61L 27/54; A61K 9/127; A61L 27/12; A61L 27/20; A61L 27/04

(52) U.S. Cl. CPC A61K 41/0052; A61L 27/20; A61L 27/52; A61L 27/042; A61L 27/54; A61K 9/127; A61L 27/12; A61F 2/28; A61L 2300/232; A61L 2300/412; A61L 2430/02; A61L 2300/112; A61F 2210/0004

(57) ABSTRACT

Provided are a composition for new bone formation and a new bone formation system, which when used to fill a bone treatment site does not effuse from the filled site due to flow of bone marrow fluid or the like, and which can promote formation of new bone at the bone treatment site. By employing a composition for new bone formation containing microparticles of a material that can emit heat in response to an external signal, and a carrier for the microparticles, formation of new bone at the bone treatment site can be promoted.

### FIELD OF THE INVENTION

The invention relates to a composition for new bone formation suited to the formation of new bone at a site requiring treatment (sometimes referred to hereinafter as “bone treatment site”), such as a bone fracture, delayed healing after a bone fracture, bone defect, bone metastasis, or the like, and a new bone formation system using the composition for new bone formation.

### DESCRIPTION OF THE RELATED ART

Bone inherently has flexibility, elasticity, and plasticity. Healthy bone is difficult to fracture, but a bone fracture occurs when repeated external force or strong external force exceeding a limit is applied. Even slight external force can sometimes also result in a bone fracture due to reduced strength of the affected site when a lesion such as a tumor is present in the bone. In particular, osteoporosis and bone fragility fractures are currently on the rise as society ages.

The locking plate method that fits a metal plate to the fracture site and fixes the plate to the bone by screws and the intramedullary nail fixation method that places a long metal rod from the bone end into the medullary cavity in the center of the bone and fixes the rod by screws by surgical operation are commonly used as methods of treating a fracture site.

Allogenic bone grafting that selects a bone corresponding to a site of damage due to a bone fracture, delayed healing of a bone fracture, or a bone defect after removing a lesion such as a tumor, from a bone bank and implants it, recycled autologous bone grafting that removes the patient's own damaged bone from the body and returns it to the body after treatment by radiation, heat treatment, or the like, autologous bone grafting that implants the patient's own bone from another site, and artificial bone grafting by hydroxyapatite and the like are known as other surgical operations.

In addition to the above surgical operations, a method of strengthening the fracture site while preventing bone cement from leaking outside the bone from the fill site and entering blood vessels by filling by surrounding a bone cement by a bioabsorbable material comprising one selected from a fibrin sheet, collagen sheet, or a homopolymer or copolymer of polylactic acid or polyglycolic acid is known (see Patent Document 1).

“Bone induction ability,” “bone conduction ability,” and “cells” are necessary for new bone formation. However, the problem is that the above methods that implant metal plates or allogenic bone or autologous recycled bone to kill the level of osteoblasts only mechanically support the fracture site and do not proactively promote new bone formation. The method of filling by surrounding a bone cement by a bioabsorbable material also is intended to prevent the bone cement from leaking outside the bone from the fill site and does not proactively form new bone. As for artificial bone grafting by hydroxyapatite and the like, artificial bone is thought to have the abovementioned bone conduction ability, but does not proactively induce bone formation by influencing osteoblasts and the like.

On the other hand, methods of implanting cultured osteoblasts, methods of implanting a new bone formation material including bone morphogenetic protein (BMP), and the like are known as methods for proactively promoting new bone formation at a bone treatment site (see Patent Document 2). These methods, however, require preparation of biological materials such as cells and protein for new bone formation, resulting in problems with time and cost.

In addition to the above methods that implant osteoblasts and new bone formation material, an ultrasound therapy instrument intended to promote formation of new bone tissue by applying ultrasonic vibration is known (see Patent Document 3). This method of applying ultrasonic vibration, however, applies ultrasonic waves from an ultrasound therapy device outside the body. The problem is that it is difficult to apply the ultrasonic waves to the bone treatment site alone, and there is a risk of exerting negative effects on other body tissues.

It is known regarding new bone formation at a bone treatment site that (1) temperature elevation of the bone treatment site is closely related to the formation of new bone (see Non-patent Document 1), (2) alkaline phosphatase (ALP) activity rises and calcification is enhanced earlier than usual when bone marrow stem cells are heated (see Non-patent Document 2), (3) the WNT/β-catenin signal, which is especially important in controlling osteoblast formation and activity and is a central pathway for adjusting the growth and maintenance of bone, is activated at 43.7° C-47.5° C. (see Non-patent Document 3). However, the above non-patent documents are the results of in vitro studies. Therefore, the bone treatment site alone must be warmed without affecting normal cells in the body for new bone formation at the bone treatment site. Nonetheless, a method of doing this is not known.

A method of bonding liposomes containing antibody-bonded magnetic microparticles to a tumor region and heating by an alternating electric field (AMF) is known as a method of warming only a specific tumor region without affecting normal cells in the body (see Patent Document 4). This method, however, requires a complex procedure to prepare an antibody that bonds specifically to the tumor cells, and it is difficult to keep the liposomes containing antibody-bonded magnetic microparticles at the bone treatment site due to the flow of bone marrow fluid and/or blood within the bone even if the bone treatment site is filled with liposomes containing antibody-bonded magnetic microparticles.

### PRIOR ARTS LIST

### Patent Documents

Patent Document 1: Japanese Unexamined Patent Application No. 2000-262609

Patent Document 2: Japanese Unexamined Patent Application No. 2012-16517

Patent Document 3: Japanese Unexamined Patent Application No. 2008-295548

Patent Document 4: Japanese Unexamined Patent Application No. 2006-273740

### Non-Patent Documents

Non-patent Document 1: Doyle JR et al., “Stimulation of bone growth by short-wave diathermy,” J. Bone Joint Surg.-Am 1963, Vol. 45, No. 1, p. 15, p. 15-24.

Non-patent Document 2: Jing Chen et al., “Enhanced Osteogenesis of Human Mesenchymal Stem Cells by Periodic Heat Shock in Self-Assembling Peptide Hydrogel,” Tissue Engineering Part A, 2013, Vol. 19, No. 5 and 6, p. 716-728.

Non-patent Document 3: Olkku et al., “Ultrasound-induced activation of Wnt signaling in human MG-63 osteoblastic cells,” Bone, 2010, Vol. 47, No. 2, p. 320-330.

### SUMMARY OF THE INVENTION

### Problems to be Solved by the Invention

The present invention is an invention intended to solve the above problems of the prior art. It was newly discovered upon in-depth research that filling a bone treatment site with microparticles including a material that can emit heat in response to an external signal (the “material that can emit heat in response to an external signal” is also referred to hereinafter as “heat-emitting material,” and the “microparticles including a material that can emit heat in response to an external signal” are also referred to hereinafter as “microparticles”) together with a carrier makes it possible to decrease outflow of microparticles from the fill site due to the flow of bone marrow fluid and the like, and that the warming effect caused by applying an external signal to these microparticles can promote new bone formation at the bone treatment site, and the present invention was perfected.

Specifically, the purpose of the present invention is to provide a composition for new bone formation and a new bone formation system.

### Means to Solve the Problems

The present invention relates to the composition for new bone formation and the new bone formation system described below.

(1) A composition for new bone formation comprising microparticles including a material that can emit heat in response to an external signal and a carrier of these microparticles.

(2) The composition for new bone formation according to (1) above, wherein the carrier is at least one selected from a gel, artificial bone, and bone cement.

(3) The composition for new bone formation according to (1) or (2) above, wherein the material is at least one selected from magnetite and maghemite.

(4) The composition for new bone formation according to any one of (1) to(3) above, wherein the microparticles are covered by liposomes.

(5) A system for new bone formation comprising the composition for new bone formation according to any one of (1) to (4) above and an external signal generator for generating an external signal to cause the microparticles contained in the composition for new bone formation to emit heat.

### Effect of the Invention

Outflow of the microparticles from the bone treatment site due to the flow of bone marrow fluid and the like can be decreased by filling the bone treatment site with microparticles together with a carrier. Therefore, a warming effect can be exerted on the fine particle-filled bone treatment site alone, and new bone formation can be promoted without imposing any burden on other body tissues.

In addition, since the microparticles are filled together with a carrier, there is no need to adsorb antibodies or the like onto the microparticles, and the microparticles can be produced by an easy method.

Furthermore, when artificial bone is used as the carrier, new bone formation by artificial bone can also be promoted in addition to promoting the development of new bone by the warming effect of the microparticles.

Since the heat-emitting material of the present invention emits heat in response to an external signal, the temperature, heat emission time, and the like of the microparticles can be adjusted by adjusting the heat-emitting material and/or the external signal. Therefore, the magnitude of the warming effect can be adjusted in accordance with the symptoms.

### BRIEF DESCRIPTION OF THE DRAWINGS

FIG. 1 shows the procedure for filling the medial side of the proximal tibia of the rat with the composition for new bone formation of the present invention;

FIG. 2 is a photograph substituted for a drawing showing radiographs of the bone 0, 2, 4, and 6 weeks after filling a burr hole with the composition for new bone formation of the present invention with and without warming;

FIG. 3 is a photograph substituted for a drawing showing radiographs for measuring the degree of bone hardening by image evaluation;

FIG. 4 is a graph showing the results of measuring the degree of bone hardening by image evaluation of radiographs taken in Example 3 and Comparative Example 2;

FIG. 5 is a photograph substituted for a drawing showing tissue cross-section photographs of the bone of a rat at a site filled by the composition for new bone formation in Example 4 and Comparative Example 3;

FIG. 6 is a photograph substituted for a drawing. FIG. 6(1) is an enlarged photograph of a cross-section of REGENOS, and FIG. 6(2) is a photograph of a composition for new bone formation produced in Example 5;

FIG. 7 is a photograph substituted for a drawing showing radiographs of Examples 6-8 and Comparative Example 5;

FIG. 8 is a graph showing the results of measurement of the degree of bone hardening by image evaluation of radiographs taken in Example 9 and Comparative Example 6;

FIG. 9 is 20× enlarged photographs of cross-sections of bone taken in Examples 10-12 and Comparative Example 7.

### DESCRIPTION OF THE PREFERRED EMBODIMENTS

The composition for new bone formation and new bone formation system of the present invention are explained in detail below.

First, the composition for new bone formation of the present invention includes microparticles including a material that can emit heat in response to an external signal and a carrier of these microparticles. The term “external signal” in the present invention means one that makes it possible for the material to emit heat upon application of this external signal to the material. Examples include a magnetic field, electrical field, ultrasonic waves, light, and the like.

The phrase “material that can emit heat in response to an external signal” in the present invention means a material that can itself emit heat upon application of the abovementioned “external signal.” The heat-emitting material is not particularly restricted as long as it can emit heat in response to an external signal, but it preferably is a material having a fast heating rate, that is stable even during heat emission, and that has no or substantially no effects on the body. From this viewpoint, examples include gold, silver, platinum, palladium, iridium, aluminum, copper, nickel, iron, magnesium, titanium, zirconium, or alloys containing two or more of these metals, as well as derivatives thereof, and the like. Moreover, metal compounds are also included in such derivatives. An example of microparticles including a heat-emitting material is microparticles including at least one of the above metals, alloys, or derivatives thereof.

In addition, an example of microparticles is magnetic microparticles composed mainly of iron having, for example, a metal fraction of 70 wt % or more, and 80 wt % or more of this metal fraction being Fe. Concrete examples of such magnetic microparticles include magnetic microparticles comprising Fe-Co, Fe—Ni, Fe—Al, Fe—Ni—Al, Fe—Co—Ni, Fe—Ni—Al—Zn, Fe—Al—Si, and other such alloys or metal compounds. Examples also include iron oxide-based ferromagnetic microparticles represented by FeOx (4/3≦x≦3/2); iron oxide microparticles having a divalent metal such as Cr, Mn, Co, or Ni added to FeOx, Co-coated FeOx microparticles having Co coated on FeOx; and chromium dioxide or oxide microparticles having a metal such as Na, K, Fe, or Mn or an oxide of these metals added to chromium dioxide. The microparticles may have any shape as long as they achieve the effects of the present invention. Examples include round, rod-type, needle-shaped, hollow element, layered structure of different metals (core-shell structure), tube-type, and the like. They may also be irregular and have protrusions.

The size of the microparticles is not particularly restricted as long as they can fill the bone treatment site together with the carrier described below. For example, when a bone treatment site is filled by a composition for new bone formation including these microparticles by syringe, the size of the microparticles should be smaller than the inner diameter of the syringe. When a bone treatment site is filled by adsorbing these microparticles onto a solid carrier described below, the size should make adsorption to the solid carrier possible and may be adjusted as is appropriate to the filling method and the like. These microparticles may be produced by known production methods, and commercial products may be used.

Since these microparticles may exhibit a warming effect when used to fill a bone treatment site together with a carrier described below and heated by an external signal, microparticles produced from only a heat-emitting material can also accomplish the effects of the present invention. However, as was mentioned above, the fluid force of bone marrow fluid and the like develops at the bone treatment site, and there is a possibility that the microparticles will detach from the carrier. In such cases, the microparticles may be coated by a material that adsorbs easily to bone tissue, for example, to keep the microparticles around the bone treatment site. Examples of materials that adsorb easily to bone tissue include liposomes, bisphosphonates, and the like. These may be coated individually or in combination. An antibody that adsorbs specifically to osteocytes may also be bonded to the microparticles or to the material coating the microparticles. On the other hand, the microparticles can also be coated by a material having a high affinity for the carrier. For example, they may be coated by an amino acid, protein, lipid, sugar, or the like having high adsorptivity to the hydroxyapatite comprising the carrier described below.

The method of coating the microparticles by a material that adsorbs easily to bone tissue may be selected as is appropriate in accordance with the type of microparticles and type of material that adsorbs easily to bone tissue. For example, the following procedure can be used when coating magnetic microparticles by liposomes. Excess ionic components are removed by washing magnetic microparticles thoroughly by deionized water, and a solution of magnetic microparticles dispersed in water is produced by sonication. Next, a phospholipid film is produced on the inner walls of an eggplant-shaped flask from a lipid mixture comprising phosphatidylcholine/phosphatidylethanolamine and N-(6-maleimidecaproyloxy)-dipalmitoylphosphatidylethanolamine. The magnetic microparticle solution produced by the above method is added to this phospholipid film, and the film is swollen while vortexing. The swollen film and the magnetic microparticles are subjected to sonication for 15 minutes. Physiological saline solution (PBS) is then added to create a state of dispersion in physiological saline solution. Conducting further sonication makes it possible to obtain magnetite liposomes.

The term “carrier” included in the composition for new bone formation of the present invention means a substance capable of filling the bone treatment site with microparticles and keeping the microparticles at the fill site. Examples include a gel, artificial bone, bone cement, and the like.

A medical gel, for example, can be used as a gel. Examples include gels comprising a combination of two or more natural organic polymers selected from alginate gel, hyaluronic acid gel, phosphatidylethanolamine-bonded polysaccharide derivative, mannan gel, and carrageenan, locust bean gum, glucomannan, starch, curdlan, guar gum, agar, cassia gum, dextran, amylose, gelatin, pectin, xanthan gum, tara gum, and gellan gum, and the like.

Examples of artificial bone include biocompatible calcium phosphate-based materials. In addition, since calcium phosphate-based materials can be stored in solid form (typically in the form of a powder) until being subjected to curing treatment, the composition for new bone formation of the present invention can be used as a preprepared type.

Calcium phosphate of various chemical compositional ratios can be included as calcium phosphate-based materials. Preferred examples include hydroxyapatite (Ca₁₀(PO₄)₆(OH)₂) or compounds that can produce hydroxyapatite by hydrolysis. Examples include mixtures having α-type tricalcium phosphate (α-Ca₃(PO₄)₂) as the main component with other calcium phosphate-based compounds as secondary components. Examples include hydroxyapatite, β-type tricalcium phosphate β-Ca₃(PO₄)₂), tetracalcium phosphate (Ca₄(PO₄)₂O), calcium hydrogenphosphate (CaHPO₄·2H₂O), and the like added to α-type tricalcium phosphate. Moreover, calcium phosphate-based compounds other than these examples may be used, and the combination of compounds used is not particularly restricted as long as the combination is capable of forming a hydroxyapatite or other calcium phosphate-based substrate (cured product).

Compounds other than the calcium phosphate-based compound that serves as the main component may be contained as well as long as a calcium phosphate-based substrate (cured product) is obtained. For example, a compound in which part of the Ca in a calcium phosphate-based compound has been substituted by another element (for example, Sr, Ba, Mg, Fe, Al, Na, K, H) may be contained. Alternatively, a compound in which part of the PO₄ has been substituted by another acid component (for example, CO₃, BO₃, SO₄, SiO₄) may be contained. Moreover, the form when using artificial bone as a carrier may be a paste or a porous solid; the form and the like are not particularly restricted as long as the microparticles can be kept at the bone treatment site. For example, when the artificial bone is a solid porous material, the microparticles may be adsorbed onto the porous material by impregnating the porous material with a solution in which the microparticles have been dispersed, or the microparticles may be kneaded into the material of the porous material during the production process of the porous material and the microparticles dispersed in the porous material produced.

A bone cement material having polymethyl methacrylate as the main component (for example, a cement material including barium powder, methyl methacrylate (monomer), and the like in addition to polymethyl methacrylate) can be used as a bone cement.

The carrier used in the present invention may be produced by adjusting the above materials as is appropriate, and commercial products may be used. For example, Atelocollagen Implant (Koken Co., Ltd.), Zyplast (Collagen Co.), Puredent (hyaluronic acid gel, Press Japan Co.), and the like are marketed as medical gels. Bone cements having methyl methacrylate as the main component are also marketed under trade names such as Surgical Simplex (manufactured by Stryker Corp.), Ostron II (manufactured by GC Corp.), and the like. Artificial bone is marketed under trade names such as Biopex-R (advance full set) manufactured by Hoya Co., Ltd.), Sera Paste (manufactured by NTK Technical Ceramics, sold by: Kobayashi Medical), Primafix (manufactured by Japan MDM Co., Ltd.), OSferion (manufactured by Olympus Co., Ltd.), REGENOS (Kuraray Co., Ltd.), and the like. The above gels, artificial bone, and bone cements may each be used individually and may be used in combinations such as gel and artificial bone, gel and bone cement, artificial bone and bone cement, and the like.

In addition, the microparticles and carrier may be included when filling the bone treatment site with the composition for new bone formation. In brief, when a gel is used as the carrier, the form of use may be any such as (1) filling the bone treatment site by microparticles dispersed in the gel beforehand, (2) filling after dispersing the microparticles in the gel at the time of filling the bone treatment site, (3) gelling by adding water or the like to the uncrosslinked material for gelation at the time of treatment, then dispersing the microparticles and filling the bone treatment site, and the like. In the case of bone cement or artificial bone as well, the form of use may be any such as (1) filling the bone treatment site with the microparticles dispersed in or adsorbed to a paste or a solid of a bone cement or artificial bone beforehand, (2) filling after dispersing or adsorbing the microparticles to a paste or a solid of a bone cement or artificial bone at the time of filling the bone treatment site, (3) making a paste of the material for the bone cement or artificial bone at the time of filling the bone treatment site, then dispersing the microparticles and filling the bone treatment site, and the like. Therefore, the “carrier” in the present invention is not restricted as to the form of use at the time of filling, and materials which may take on the forms thereof are also included.

The external signal of the present invention preferably has no effect or little effect on the party being administered. The use of a magnetic field (changes in magnetic field) or light is preferred from this viewpoint. An alternating magnetic field, for example, can be used as a magnetic field, and the intensity and duration of the magnetic field may be adjusted as is appropriate to achieve the desired temperature in accordance with the type of heat-emitting material used. For example, a magnetic field of 50 kHz-10 MHz may be applied in the case of microparticles of about 10 nm-100 nm of magnetite (Fe₃O₄) or maghemite (γ-Fe₂O₃), which are thought to cause virtually no harm to the human body, or these microparticles coated by liposomes or the like. The warming efficiency deteriorates with magnetic fields less than 50 kHz, and water and blood are also heated with magnetic fields greater than 10 MHz, which is undesirable.

In the case of light, the wavelength and the like may be selected in accordance with the type of heat-emitting material used and may be adjusted as is appropriate to achieve the desired temperature. For example, in the case of microparticles using gold as a heat-emitting material, it is preferable to use light of from 800 nm to 1200 nm, which causes virtually no harm to tissues or cells in an animal body. When the heat-emitting material is gold, it is also possible to provide from 10 MHz in the microwave region from short waves to 2 GHz high frequency waves, and the application time may be determined as is appropriate in accordance with the frequency used in application.

A known alternating magnetic field generator, Thermotron RF-8, lamp or YAG laser, or microwave generator may be used as the external signal generator for applying the abovementioned external signal included in the new bone formation system of the present invention.

In addition, the temperature of the microparticles is preferably a temperature capable of activating the Wnt/β-catenin signal. Warming to 42.5° C.-47° C. is preferred, and warming to 44° C.-46° C. is more preferred. Temperatures lower than 42.5° C. do not activate the Wnt/β-catenin signal, and temperatures higher than 47° C. risk killing normal cells of the body adjacent to the fill site, which is undesirable.

The bone treatment site to which the composition for new bone formation of the present invention can be applied is not particularly restricted as long as it is a site that requires new bone formation, such as a site that has suffered a bone fracture due to physical pressure, a site where healing of a bone fracture is delayed, a site having a defect due to removal of a tumor or the like, a site where the bone has become fragile due to a metastatic bone lesion, or the like.

When using the composition for new bone formation of the present invention, an incision may be made in the body by a surgical operation and the bone treatment site filled with the composition for new bone formation, or the bone treatment site may be filled with the composition for new bone formation from outside the body using a syringe or the like, an external signal is applied to the microparticles by an external signal generator, and a warming effect is actualized by heat emission.

Examples appear below, and the present invention is explained concretely. However, these examples are merely provided as references for concrete embodiments to explain the present invention. These illustrations are intended to explain specific concrete embodiments of the present invention, but do not limit or represent limitations to the scope of the invention disclosed in this application.

### EXAMPLES

### Example 1

### Production of Liposome-Coated Microparticles

A solution of magnetite dispersed in water was produced by washing magnetic microparticles (10 nm magnetite: manufactured by Toda Kogyo Co., Ltd.) thoroughly with deionized water to remove the excess ionic components and conducting sonication. A phospholipid film was produced on the inner walls of an eggplant-shaped flask from a lipid mixture comprising phosphatidylcholine/phosphatidylethanolamine (ratio, 2:1) and N-(6-maleimidecaproyloxy)-dipalmitoylphosphatidylethanolamine. The magnetite solution produced by the above method was added to this phospholipid film, and the film was swollen while vortexing. The swollen film and magnetic microparticles were sonicated (28 W) for 15 minutes, and 200 μL of physiological saline solution (PBS) having a 10× concentration was then added and a state of dispersion in physiological saline solution was created. Further sonication was carried out for 15 minutes (28 W), and a solution in which magnetite liposomes (MCL) were dispersed was obtained.

### Production of a Composition for New Bone Formation

The above solution in which magnetite liposomes were dispersed (36 mg/mL) and 1.2% alginate solution (FMC manufactured by BioPolymer Co., KELTONE LVCR) were mixed in a 1:1 ratio, and a composition (gel) for new bone formation was produced.

### Implantation Into Rats, Confirmation of New Bone Formation

### Example 2

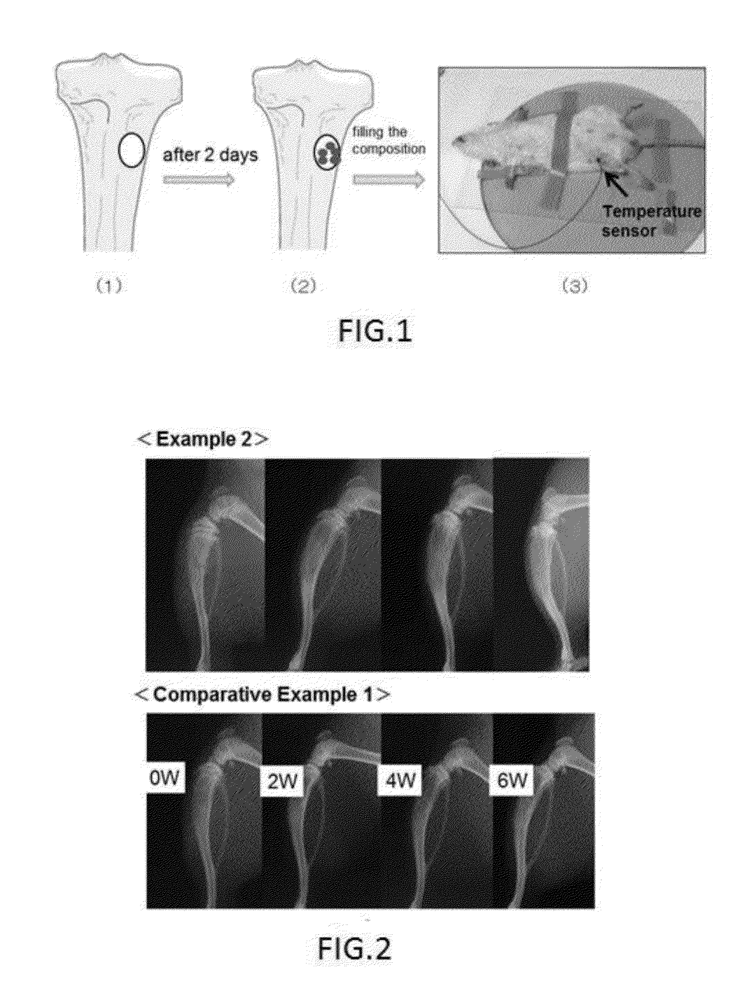

Eight-week-old rats (Sprague-Dawley rats) were anesthetized by influrane inhalation (5% concentration) and intraperitoneal administration of pentobarbital (10 mg/animal, Kyoritsu Seiyaku). Next, as shown in FIG. 1(1), a burr hole approximately 2 mm in diameter and 3 mm deep was made in the medial side of the proximal tibia of the right leg of the rat by a drill. Two days after the bleeding stopped, 100 μL of fresh composition for new bone formation produced in Example 1 was used to fill the burr hole by tweezers, as shown in FIG. 1(2). A carbon temperature sensor was placed on top when filling by the composition for new bone formation. FIG. 1(3) is a photograph of the temperature sensor set on the rat. Immediately after filling by the composition for new bone formation, the microparticles were caused to emit heat by applying a 100 KHz alternating magnetic field as an external signal using a high-frequency magnetic field generator (manufactured by Dai-ichi Koshuha Kogyo Co., Ltd.). The composition for new bone formation was kept continuously at a temperature of 45° C. for 15 minutes. Warming was conducted only once. New bone formation was evaluated by taking x-rays immediately, 2 weeks, 4 weeks, and 6 weeks after warming.

### Comparative Example 1

The experiment was conducted by the same procedure as in Example 2 except that no alternating magnetic field was applied.

FIG. 2 is the results of the radiographs of Example 2 and Comparative Example 1. As is evident from the photographs, hardening of the bone around the burr hole was confirmed after 2 weeks in Example 2. There were few findings of bone hardening after 2 weeks in the case of Comparative Example 1. These results confirmed that warming the bone treatment site promotes the development of new bone.

### Measurement of Degree of Bone Hardening by Image Evaluation

### Example 3

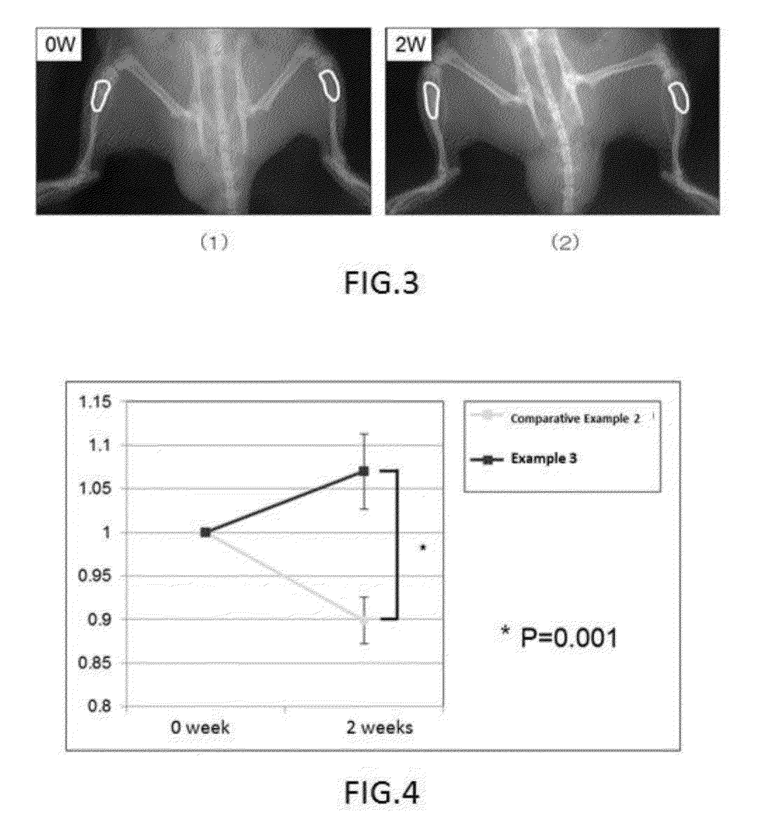

The changes over time in the degree of bone hardening were observed by measuring the average density in a certain area around the burr hole using image analysis software. First, the right leg (affected side) of a rat was filled with a composition for new bone formation and heated by the same procedure as in Example 2. Radiographs including the right leg (affected side) and healthy left leg (healthy side) were taken immediately after warming as shown in FIG. 3(1) and after 2 weeks as shown in FIG. 3(2). The radiographic images obtained were input to a computer, and the area around the burr hole of the right leg (affected side) of the radiograph taken immediately after warming was installed in the image analysis software Image J (NIH) (part surrounded by a white line in the figure), and the same area as the right leg (affected side) of the medial side of the proximal tibia of the left leg (healthy side) was also installed using a mouse in the Image J (NIH) (part surrounded by a white line in the figure). The radiographs taken after 2 weeks were also installed in the same way using a mouse in the Image J (NIH). Next, since there were virtually no changes in terms of bone formation in the left leg (healthy side), that is, virtually no changes in the radiographs, analysis was conducted to compare only the changes in the bone formation images of the right leg (affected side). The values of the right leg (affected side)/left leg (healthy side) at week 0 were taken as 1 and graphed together with the values of the right leg (affected side)/left leg (healthy side) after 2 weeks. Fourteen rats in which the composition for new bone formation was filled and heated were prepared, and the average values were used.

### Comparative Example 2

Analysis and graphing were conducted by the same procedure as in Example 3 except that warming was not performed. Seven control rats were prepared, and the average values were used.

FIG. 4 is a graph showing the results of Example 3 and Comparative Example 2. In Example 3, bone formation (density) of the right leg (affected side) was clearly elevated in comparison to the left leg (healthy side). On the other hand, in Comparative Example 2, bone formation (density) of the right leg (affected side) was instead decreased in comparison to the left leg (healthy side). This is thought to be because the microparticles themselves appear white due to x-ray absorption immediately after filling by the composition for new bone formation, but the x-ray appears black after 2 weeks since the microparticles have flowed and the residual amount was decreased. The above results confirmed that a new bone formation system including the composition for new bone formation of the present invention forms new bone at the bone treatment site.

### Histological Evaluation

### Example 4

Filling and warming of a composition for new bone formation were conducted by the same procedure as in Example 2 except that the warming temperature was 46° C. Two weeks later, the leg filled by the composition for new bone formation was removed, fixed by paraformaldehyde, made into 5 μm thick slices, and stained by hematoxylin-eosin. Next, stained slices of the site filled by the composition for new bone formation and heated were photographed using an optical microscope. The new bone formation images around the magnetite were evaluated under the microscope.

### Comparative Example 3

The cross-section of the bone of the site filled by the composition for new bone formation by the same procedure as in Example 4 except that warming was not conducted was photographed.

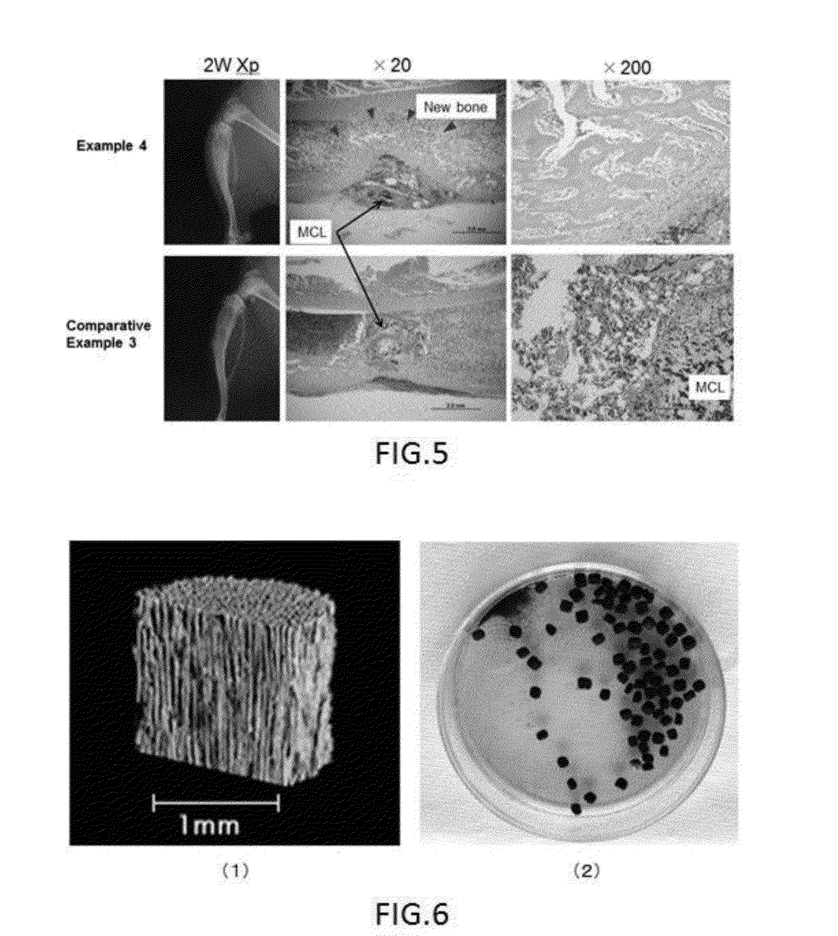

FIG. 5 shows radiographs taken before removal of the leg in Example 4 and Comparative Example 3 and enlarged photographs of the cross-section of the bone. As is evident from the photographs, a large amount of new bone was found around the burr hole in Example 4, but only new bone bordering the microparticles was found in Comparative Example 3.

### Filling by only MCL

### Comparative Example 4

Burr holes were filled with only the MCL produced in Example 1 by the same procedure as in Example 2. When the temperature was observed by carbon temperature sensor while applying an alternating magnetic field, complete diffusion of the MCL inside the bone was confirmed.

### Example 5

### Production of a Composition for New Bone Formation

A dish containing 330 mg of REGENOS (Kuraray Co., Ltd.), an artificial bone, in a solution in which the magnetite liposomes (MCL) produced in [Production of liposome-coated microparticles] of Example 1 were dispersed was placed in a negative pressure generator (GCD-051XA manufactured by TAITEX). Next, a composition for new bone formation (porous solid) having magnetite liposomes adsorbed inside the pores of the REGENOS was produced by treatment for 60 minutes under 0.067 Pa negative pressure. FIG. 6(1) is an enlarged photograph of a REGENOS cross-section; FIG. 6(2) is a photograph of the composition for new bone formation produced in Example 5.

### Implantation Into Rats, Confirmation of New Bone Formation

### Example 6

Burr holes were produced by the same procedure as in Example 2. Two days after the bleeding stopped, the burr hole was filled by one piece of the composition for new bone formation produced in Example 5 using tweezers. Next, warming was conducted by the same procedure as in Example 2, and an evaluation was made by taking radiographs immediately and 2 weeks after warming. Two rats were used in evaluation.

### Example 7

Evaluation was performed by the same procedure as in Example 6 except that the warming temperature was 44° C.

### Example 8

Evaluation was performed by the same procedure as in Example 6 except that the warming temperature was 43° C.

### Comparative Example 5

Evaluation was performed by the same procedure as in Example 6 except that warming was not conducted.

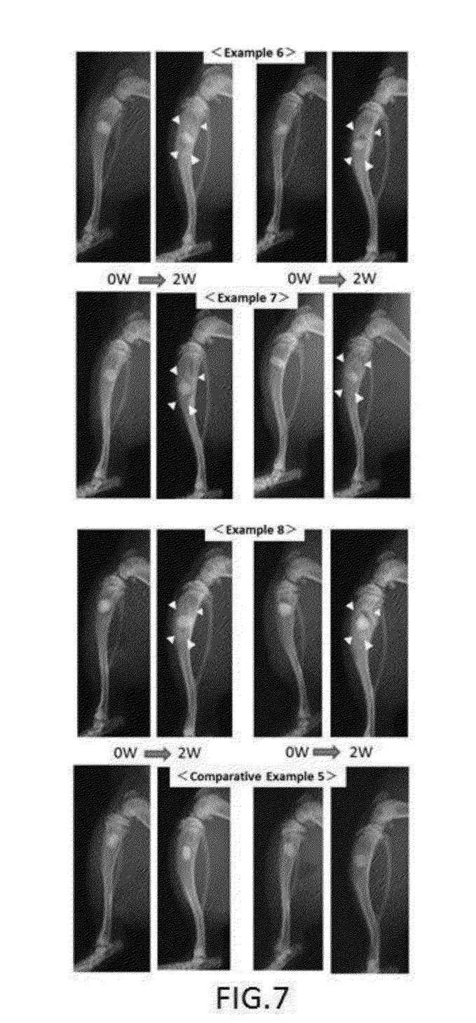

FIG. 7 shows the radiographs of Examples 6-8 and Comparative Example 5. As is evident from the photographs, hardening of the bone around the burr holes was confirmed after 2 weeks in Examples 6-8 (A in the figures), but there were few findings of bone hardening after 2 weeks in the case of Comparative Example 5. The above results confirmed that new bone formation is promoted by warming the bone treatment site when artificial bone is used as a carrier as well.

### Measurement of Degree of Bone Hardening by Image Evaluation

### Example 9

Analysis and graphing were conducted by the same procedure as in Example 3 except that the composition for new bone formation produced in Example 5 was used. Moreover, 12 rats were prepared, and the average values were used.

### Comparative Example 6

Analysis and graphing were conducted by the same procedure as in Example 9 except that warming was not performed. Three control rats were prepared, and the average values were used.

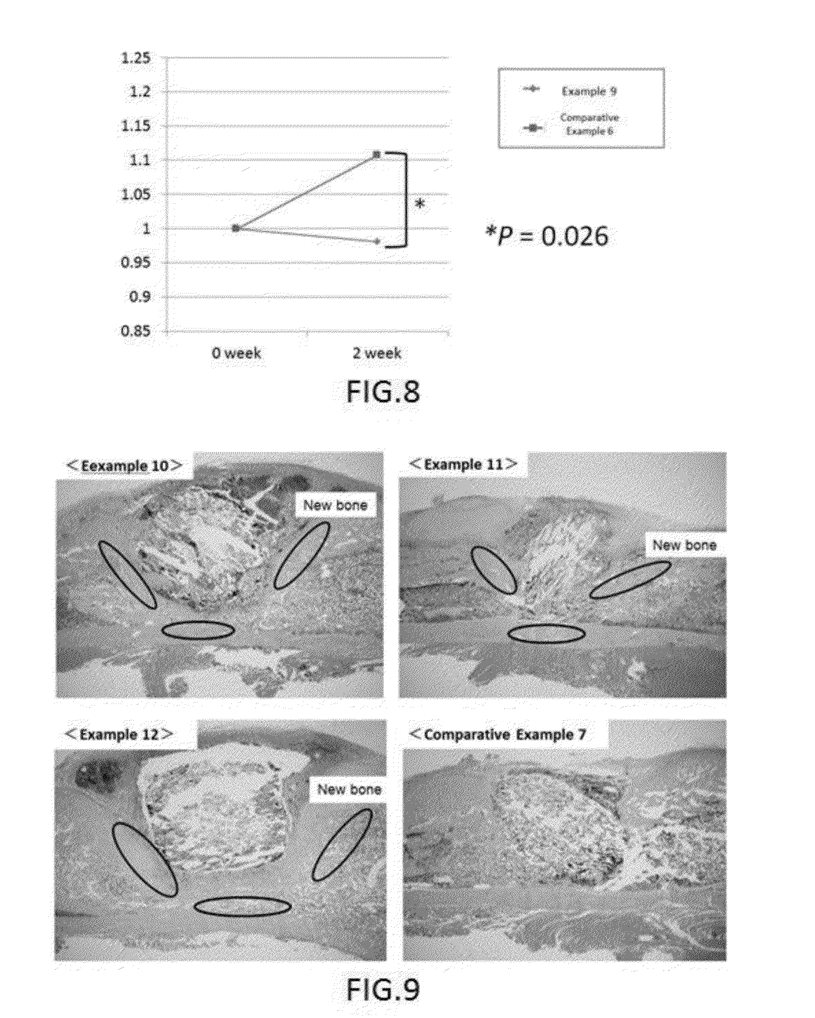

FIG. 8 is a graph showing the results of measurement of the degree of bone hardening by image evaluation of radiographs taken in Example 9 and Comparative Example 6. In Example 9, bone formation (density) of the right leg (affected side) was clearly elevated in comparison to the left leg (healthy side). On the other hand, in Comparative Example 6, bone formation (density) of the right leg (affected side) was instead decreased in comparison to the left leg (healthy side). This is thought to be because the microparticles themselves appear white due to x-ray absorption immediately after filling by the composition for new bone formation. Moreover, the decrease in bone formation (density) in Comparative Example 6 was thought to be less than in Comparative Example 2 because fewer microparticles were lost due to flow than when a gel was used as a carrier. The above results confirmed that a new bone formation system including the composition for new bone formation of the present invention forms new bone at the bone treatment site.

### Histological Evaluation

### Example 10

Cross-sections of the bone of the site filled by the composition for new bone formation by the same procedure as in Example 4 were photographed and evaluated using one rat after the radiographs had been taken in Example 6.

### Example 11

Cross-sections of the bone of the site filled by the composition for new bone formation by the same procedure as in Example 10 were photographed, except that a rat from Example 7 was used.

### Example 12

Cross-sections of the bone of the site filled by the composition for new bone formation by the same procedure as in Example 10 were photographed, except that a rat from Example 8 was used.

### Comparative Example 7

Cross-sections of the bone of the site filled by the composition for new bone formation by the same procedure as in Example 10 were photographed, except that a rat from Comparative Example 5 was used.

FIG. 9 shows 20× enlarged photographs of cross-sections of the bone photographed in Examples 10-12 and Comparative Example 7. As is evident from the photographs, a large amount of new bone was found around the burr holes in Examples 10-12, but only new bone bordering the microparticles was found in Comparative Example 7.

### INDUSTRIAL APPLICABILITY

The use of the composition for new bone formation of the present invention makes it possible to promote the development of new bone when treating a bone fracture or the like. In addition, since it does not require the use of an antibody or the like and can be used together with a bone cement or artificial bone and medical gel and the like used in the past in the treatment of bone fractures, it can be utilized as a treatment material for bone fracture sites in hospitals, emergency centers, and other such medical facilities, university medical departments and other such research facilities, general hospitals, and the like.

1-5. (canceled)

6. A composition for new bone formation for promoting the formation of new bone by heating after filling a bone treatment site, including microparticles including a material that can emit heat in response to an external signal and a carrier of said microparticles, wherein the carrier does not include at least one of albumin and collagen.

7. The composition for new bone formation according to claim 6, characterized in that the bone treatment site is selected from among a site that has suffered a bone fracture due to physical pressure, a site where healing of a bone fracture is delayed, a site having a defect due to removal of a tumor or the like, a site where the bone has become fragile due to a metastatic bone lesion or the like.

8. The composition for new bone formation according to claim 6, wherein the carrier is at least one selected from a gel, artificial bone, and bone cement.

9. The composition for new bone formation according to claim 6, wherein the material is at least one selected from magnetite and maghemite.

10. The composition for new bone formation according to claim 8, wherein the material is at least one selected from magnetite and maghemite.

11. The composition for new bone formation according to claim 6, wherein the microparticles are covered by liposomes.

12. The composition for new bone formation according to claim 8, wherein the microparticles are covered by liposomes.

13. The composition for new bone formation according to claim 9, wherein the microparticles are covered by liposomes.

14. A system for new bone formation comprising the composition for new bone formation according to claim 6 and an external signal generator for generating an external signal to cause the microparticles contained in the composition for new bone formation to emit heat.

15. A system for new bone formation comprising the composition for new bone formation according to claim 8 and an external signal generator for generating an external signal to cause the microparticles contained in the composition for new bone formation to emit heat.

16. A system for new bone formation comprising the composition for new bone formation according to claim 9 and an external signal generator for generating an external signal to cause the microparticles contained in the composition for new bone formation to emit heat.

17. A system for new bone formation comprising the composition for new bone formation according to claim 10 and an external signal generator for generating an external signal to cause the microparticles contained in the composition for new bone formation to emit heat.

18. A system for new bone formation comprising the composition for new bone formation according to claim 11 and an external signal generator for generating an external signal to cause the microparticles contained in the composition for new bone formation to emit heat.

19. A system for new bone formation comprising the composition for new bone formation according to claim 12 and an external signal generator for generating an external signal to cause the microparticles contained in the composition for new bone formation to emit heat.

20. A method for promoting the formation of new bone at a bone treatment site, characterized in including a step for filling a bone treatment site with the composition for new bone formation according to claim 6 and a step for causing the microparticles included in the composition for new bone formation to emit heat in response to an external signal.

21. The method for promoting the formation of new bone at a bone treatment site according to claim 20, characterized in that the bone treatment site is selected from among a site that has suffered a bone fracture due to physical pressure, a site where healing of a bone fracture is delayed, a site having a defect due to removal of a tumor or the like, a site where the bone has become fragile due to a metastatic bone lesion or the like.

22. A method for promoting the formation of new bone at a bone treatment site, characterized in including a step for filling a bone treatment site with the composition for new bone formation according to claim 8 and a step for causing the microparticles included in the composition for new bone formation to emit heat in response to an external signal.

23. A method for promoting the formation of new bone at a bone treatment site, characterized in including a step for filling a bone treatment site with the composition for new bone formation according to claim 9 and a step for causing the microparticles included in the composition for new bone formation to emit heat in response to an external signal.

24. A method for promoting the formation of new bone at a bone treatment site, characterized in including a step for filling a bone treatment site with the composition for new bone formation according to claim 10 and a step for causing the microparticles included in the composition for new bone formation to emit heat in response to an external signal.

25. A method for promoting the formation of new bone at a bone treatment site, characterized in including a step for filling a bone treatment site with the composition for new bone formation according to claim 11 and a step for causing the microparticles included in the composition for new bone formation to emit heat in response to an external signal .
